(19) United States
(12) Patent Application Publication
(10) Pub. No.: US 2026/0283717 A1
(43) Pub. Date: Sep. 24, 2026

(54) SURGERY ASSISTANCE DEVICE, METHOD OF ASSISTING SURGERY, AND COMPUTER PROGRAM

(71) Applicant: ASAHI INTECC CO., LTD., Seto-shi (JP)

(72) Inventors: Yukari SHIBAYAMA, Seto-shi (JP); Shumpei Yoshitake, Seto-shi (JP)

(73) Assignee: ASAHI INTECC CO., LTD., Seto-shi (JP)

(21) Appl. No.: 19/239,380

(22) Filed: Jun. 16, 2025

(30) Foreign Application Priority Data
Mar. 19, 2025 (JP) 2025-044709

(51) Int. Cl. A61B 34/30; A61B 34/20; A61B 90/00

(52) U.S. Cl. CPC A61B 34/30; A61B 34/20; A61B 90/361; A61B 2034/2068; A61B 2034/303

(57) ABSTRACT

This surgery assistance device includes a two-dimensional image generation unit, a three-dimensional image generation unit, and a composite image generation unit. The two-dimensional image generation unit generates a two-dimensional image showing a placement position of an FPD in two-dimensional coordinates, and includes a first planar trajectory on which the placement position of the FPD is located in a first direction perpendicular to a target blood vessel. The three-dimensional image generation unit generates a three-dimensional image showing the placement position of the FPD in three-dimensional coordinates, and includes a blood vessel axis showing the target blood vessel as a line segment extending from the origin, and a first three-dimensional trajectory showing the first planar trajectory as a circumference of a circle having a radius of a predetermined length from the origin. The composite image generation unit generates and outputs a composite image, which combines the two-dimensional image and the three-dimensional image.

### INCORPORATION BY REFERENCE

The present application claims the benefit of Japanese Patent Application No. 2025-044709 filed on Mar. 19, 2025, the disclosure of which is incorporated by reference in its entirety.

### TECHNICAL FIELD

The present disclosure relates to a surgery assistance device, a method of assisting a surgery, and a computer program.

### BACKGROUND ART

In recent years, flat panel detectors are being used in blood vessel imaging that is carried out in examinations and therapy. Hereinafter, a flat panel detector is also referred to as an “FPD”. FPD stands for flat panel detector. An FPD is a device that obtains an image by acquiring X-rays that have been transmitted through a human body, and then converting the X-rays into a digital signal. An FPD is also referred to as a flat panel X-ray detector. Such FPDs have the advantages of being able to achieve higher-definition image quality, shorter image display times, and lower radiation exposure compared to conventional CR systems. CR stands for computed radiography. An imaging device including an FPD is also referred to as a radiation imaging device. For example, JP 2013-233413 A discloses, in a radiation imaging device, the generation of a graph that includes points indicating the recommended angle of a C-arm to which an FPD is attached.

### SUMMARY

### Technical Problem

An operator acquires an image of a blood vessel subjected to examination or therapy using an FPD, and operates a medical device while confirming the positions of each of the blood vessel, a lesion, and the medical device that has been inserted into the blood vessel included in the image. The blood vessel subjected to examination or therapy is referred to as a “target blood vessel” below. In a case where the placement position of the FPD is not appropriate, there is a problem that the operator is unable to correctly grasp the positional relationship between the target blood vessel, the lesion, and the medical device, and the medical device cannot be pushed forward in an intended direction. The medical device is a guide wire or catheter. In this respect, the technique described in JP 2013-233413 A merely displays a graph showing points indicating the recommended angle of the C-arm in two-dimensional coordinates. Therefore, it cannot be said that the placement position of the FPD is easy to grasp.

### Solution to Problem

The present disclosure has been made to solve at least part of the problem described above, and can be implemented as the following aspect.

According to an aspect of the present disclosure, a surgery assistance device is provided. The surgery assistance device includes: a two-dimensional image generation unit and a three-dimensional image generation unit that generate, in a three-dimensional space, an image showing a range of placement positions of a flat panel detector (FPD) when a target blood vessel is imaged using the FPD, which can be moved along a surface of a virtual sphere having a predetermined radius and is centered at an origin of the three-dimensional space; and a composite image generation unit that generates a composite image. The two-dimensional image generation unit generates a two-dimensional image showing the placement position of the FPD in two-dimensional coordinates, and which is a two-dimensional image including a first planar trajectory on which the placement position of the FPD is located in a first direction, which is perpendicular to an extending direction of the target blood vessel. The three-dimensional image generation unit generates a three-dimensional image showing the placement position of the FPD in three-dimensional coordinates, and is a three-dimensional image that includes a blood vessel axis that represents the extending direction of the target blood vessel as a line segment extending from the origin of the three-dimensional coordinates, and a first three-dimensional trajectory that shows the first planar trajectory as a circumference of a circle having a radius of a predetermined length from the origin of the three-dimensional coordinates. The composite image generation unit generates and outputs a composite image, in which the two-dimensional image and the three-dimensional image have been combined.

### BRIEF DESCRIPTION OF DRAWINGS

FIG. 1 is an explanatory diagram showing an example of a surgery assistance system.

FIG. 2 is a diagram that illustrates an LAO.

FIG. 3 is a diagram that illustrates an RAO.

FIG. 4 is a diagram that illustrates a CRA.

FIG. 5 is a diagram that illustrates a CAU.

FIG. 6 is a flowchart showing an example of the processing procedure of guide processing.

FIG. 7 is a diagram showing an example of a first screen in an initial state.

FIG. 8 is an explanatory diagram of an acquisition method of a target blood vessel using the first screen.

FIG. 9 is a diagram showing an example of a composite image.

FIG. 10 is a diagram showing another example of a composite image.

FIG. 11 is a diagram showing another example of a composite image.

FIG. 12 is a diagram showing an example of a composite image according to a second embodiment.

FIG. 13 is a diagram showing an example of a composite image according to a third embodiment.

FIG. 14 is a diagram showing an example of a composite image according to a fourth embodiment.

FIG. 15 is a diagram showing an example of a composite image according to a fifth embodiment.

FIG. 16 is a diagram showing an example of a composite image according to a sixth embodiment.

FIG. 17 is a diagram showing an example of a composite image according to a seventh embodiment.

FIG. 18 is a diagram showing an example of a composite image according to an eighth embodiment.

### DETAILED DESCRIPTION OF EMBODIMENTS

### First Embodiment

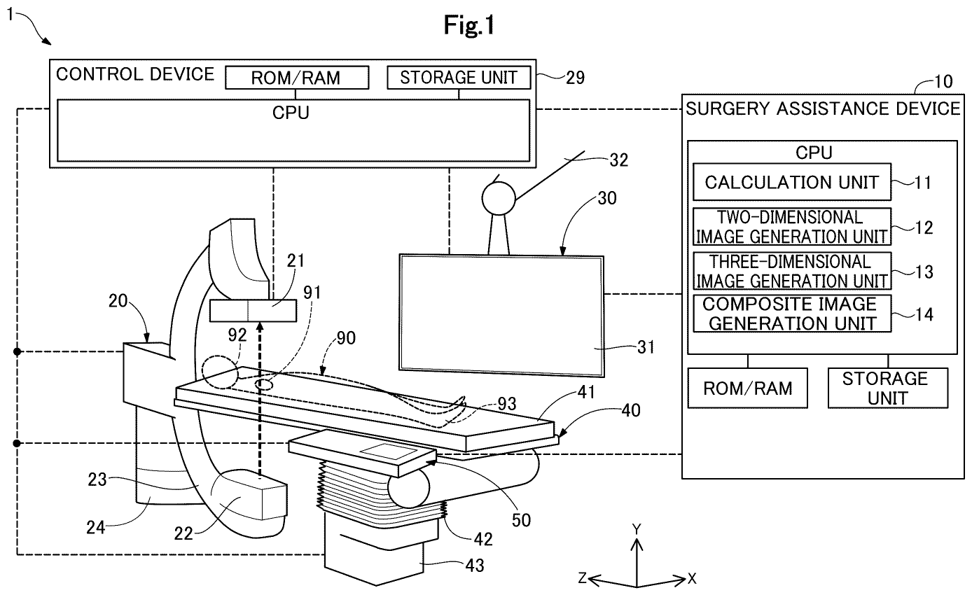

FIG. 1 is an explanatory diagram showing an example of a surgery assistance system 1. The surgery assistance system 1 is a system that acquires an X-ray image of a human body for examination or therapy, and is also referred to as a “blood vessel imaging system”. The surgery assistance system 1 includes a surgery assistance device 10, a radiation imaging device 20 having a flat panel detector, a display device 30, a table 40, and an operation unit 50. Hereinafter, a flat panel detector is also referred to as an “FPD”. FPD stands for flat panel detector. The surgery assistance system 1 according to the present embodiment is capable of guiding the range of placement positions of the FPD as a result of including the surgery assistance device 10 described below. The “target blood vessel” refers to the blood vessel subjected to examination or therapy. The surgery assistance system 1 may be used not only in the blood vessel system, but also in biological lumens such as the lymphatic system, biliary system, urinary system, respiratory system, digestive system, secretory glands, and reproductive organs.

FIG. 1 illustrates mutually orthogonal X, Y, and Z axes. The X-axis corresponds to the width direction of the radiation imaging device 20. The Y-axis corresponds to the height direction of the radiation imaging device 20. The Z-axis corresponds to the depth direction of the radiation imaging device 20. In the following description, the direction in which the head 92 of a human body 90 having the target blood vessel is also referred to as the “Z-axis direction”, and is sometimes represented as “Z”. The human body 90 represents a patient.

The surgery assistance device 10 generates, as a result of the guide processing described below, an image representing a range of placement positions of the FPD of the radiation imaging device 20, and outputs the generated image to the display device 30. The surgery assistance device 10 includes a CPU, a ROM, a RAM, and a storage unit. The surgery assistance device 10, as a result of the CPU executing a computer program that is stored in the ROM, realizes the functions of a calculation unit 11, a two-dimensional image generation unit 12, a three-dimensional image generation unit 13, and a composite image generation unit 14. The surgery assistance device 10 is electrically connected to each of a control device 29 of the radiation imaging device 20, the display device 30, and the operation unit 50. The surgery assistance device 10 transmits information to, and receives information from, the control device 29 of the radiation imaging device 20, the display device 30, and the operation unit 50.

The functions realized by the constituent elements described herein may be implemented by circuitry or processing circuitry including a general-purpose processor, an application-specific processor, an integrated circuit, an application-specific integrated circuit (ASIC), a central processing unit (CPU), a conventional circuit, and/or any combination thereof, which have been programmed to realize the described functions. The processor includes transistors and other circuits, and is regarded as circuitry or processing circuitry. The processor may be a programmed processor which executes a program stored in a memory. Herein, circuitry, units, and means are hardware programmed to realize the described functions, or hardware that executes the described functions. The hardware may be any type of hardware disclosed herein, or any type of hardware that has been programmed to achieve the described functions or known to execute the described functions. In a case where the hardware is a processor which is regarded as circuitry, the circuitry, means, or unit is a combination of hardware and software used to configure the hardware and/or the processor.

The calculation unit 11 calculates a blood vessel axis of the target blood vessel, and first to fourth planar trajectories PO1 to PO4. The two-dimensional image generation unit 12 generates an image showing the range of placement positions of the FPD when the operator uses the FPD to image the target blood vessel. The operator is a physician that performs the procedure, or a physician's assistant. The image generated by the two-dimensional image generation unit 12 is a two-dimensional image showing the placement position of the FPD in two-dimensional coordinates. The three-dimensional image generation unit 13 generates an image representing the range of placement positions of the FPD when the operator uses the FPD to image the target blood vessel. The image generated by the three-dimensional image generation unit 13 is a three-dimensional image showing the placement position of the FPD in three-dimensional coordinates. The composite image generation unit 14 generates a composite image in which the two-dimensional image that has been generated by the two-dimensional image generation unit 12 and the three-dimensional image that has been generated by the three-dimensional image generation unit 13 have been combined, and then outputs the composite image to the display device 30. These details will be described later.

The radiation imaging device 20 includes the FPD. The radiation imaging device 20 is a device that obtains an X-ray image by acquiring X-rays that have been transmitted through a human body, and then converting the X-rays into a digital signal. The X-ray image acquired by the radiation imaging device 20 is also referred to as an “angiographic image”. The radiation imaging device 20 includes the FPD 21, an X-ray tube device 22, a C-arm 23, a support portion 24, and the control device 29.

The FPD 21 includes a flat panel X-ray detector. The FPD 21 converts the X-rays that have entered from the X-ray tube device 22 into an electrical signal, performs A/D conversion, and then generates an X-ray image. A/D refers to analog/digital. The X-ray tube device 22 receives a high voltage output from an X-ray high voltage device (not shown), and irradiates an X-ray beam. As indicated by the bold dashed line in the Y-axis direction in FIG. 1, the X-ray beam irradiated from the X-ray tube device 22 enters the FPD 21 via the human body 90. The C-arm 23 is a letter C-shaped arm that fixes the FPD 21 and the X-ray tube device 22 in opposing positions. The C-arm 23 is a support that supports the FPD 21 and the X-ray tube device 22. The support portion 24 rotatably supports the C-arm 23. That is, the FPD 21 and the X-ray tube device 22 can be moved to an arbitrary imaging position around the human body 90 lying on a bed 41 while being fixed in opposing positions by the C-arm 23. The radiation imaging device 20 is also sometimes simply referred to as an “FPD” or “FPD device”.

The control device 29 includes a CPU, a ROM, a RAM, and a storage unit. In the control device 29, the CPU controls the entire radiation imaging device 20 by executing a computer program stored in the ROM. The control device 29 is electrically connected to each of the FPD 21, the support portion 24, the display device 30, the table 40, and the operation unit 50. The control device 29 displays the X-ray image generated by the FPD 21 on the display device 30. The control device 29 drives the support portion 24 and rotates the C-arm 23 according to an operation from the operation unit 50. The control device 29 changes the height of the bed 41 by extending or retracting an extending/retracting portion 42, and changes the position of the bed 41 by moving the table 40 in the Z-axis direction according to operations from the operation unit 50.

The display device 30 is connected to the surgery assistance device 10 and the control device 29. The display device 30 is an output interface to the surgery assistance device 10 and the radiation imaging device 20. The display device 30 includes a monitor 31 and an arm 32. As the monitor 31, it is possible to adopt a known means such as a liquid-crystal display, smart glasses, or a projector. The display device 30 is also referred to as a “display unit”. The arm 32 supports and fixes the monitor 31. In a case where the monitor 31 represents smart glasses or a projector, the arm 32 may be omitted.

The table 40 is a stage for the human body 90 to lie on and be positioned near the FPD 21. The table 40 includes the bed 41, the extending/retracting portion 42, and a leg portion 43. The bed 41 includes a mattress on which the human body 90 can lay down. The bed 41 is supported by the table 40 so as to be movable in the Z-axis direction. The extending/retracting portion 42 changes the height of the bed 41 by extracting and contracting in the Y-axis direction. The leg portion 43 supports the bed 41 and the extending/retracting portion 42. As indicated by the dashed line in FIG. 1, the human body 90 is laid facing upward on the bed 41, in a state where the head 92 is placed on the side closer to the FPD 21, and the feet 93 are placed on the side further away from the FPD 21. In this way, it is easy to acquire an image of the target blood vessel in the heart 91 using the FPD 21.

The operation unit 50 is connected to the surgery assistance device 10 and the control device 29. The operation unit 50 is an input interface to the surgery assistance device 10 and the radiation imaging device 20. As the operation unit 50, a known means can be adopted, such as a touch panel, an operation button, an operation lever, an operation switch, a keyboard, a mouse, a voice input unit, or a foot switch. The operation unit 50 is also referred to as an “input unit”. In the illustrated example, the operation unit 50 is fixed to the table 40.

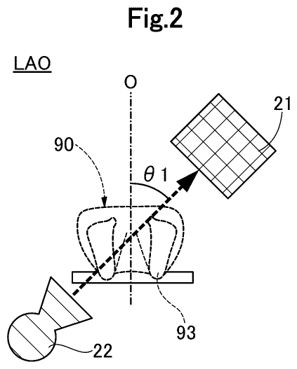

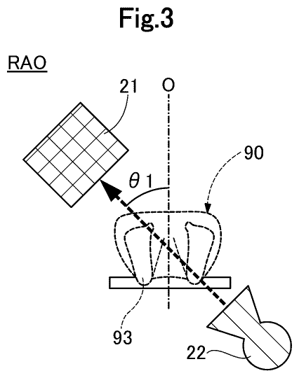

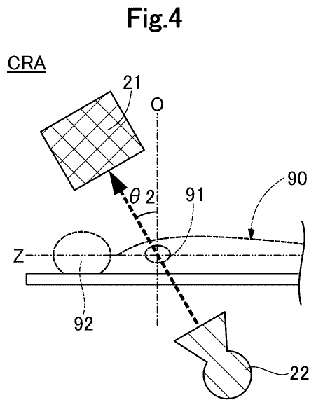

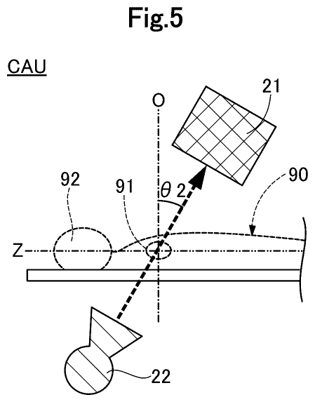

FIG. 2 is a diagram that illustrates an LAO. FIG. 3 is a diagram that illustrates an RAO. FIG. 4 is a diagram that illustrates a CRA. FIG. 5 is a diagram that illustrates a CAU. The placement position of the FPD 21 will be described using FIGS. 2 to 5. The placement position of the FPD 21 is, in other words, the placement position of the C-arm 23. As shown in FIG. 2, a case where the FPD 21 is positioned in the left direction of the human body 90 is referred to as an LAO. LAO stands for left anterior oblique view. As shown in FIG. 3, a case where the FPD 21 is positioned in the right direction of the human body 90 is referred to as an RAO. RAO stands for right anterior oblique view. As shown in FIG. 4, a case where the FPD 21 is positioned in the upper direction of the human body 90 is referred to as a CRA. CRA stands for cranial. As shown in FIG. 5, a case where the FPD 21 is positioned in the lower direction of the human body 90 is referred to as a CAU. CAU stands for caudal. That is, the “placement position of the FPD 21” is specified by a combination of a left-right position a1 and an up-down position a2 as described below:
(a1) LAO or RAO, and an angle θ1 from the center O of the human body 90; and
(a2) CRA or CAU, and an angle θ2 from the center O of the human body 90.
For example, “RAO 28 CRA 5” means that the FPD 21 is positioned at a 28 degrees angle in the right direction of the human body 90, and is positioned at a 5 degree angle in the upper direction of the human body 90.

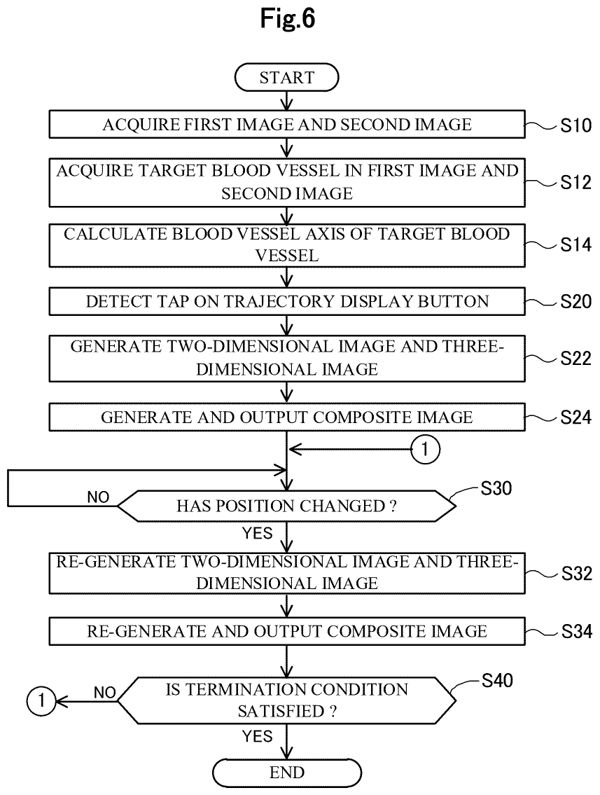

FIG. 6 is a flowchart showing an example of the processing procedure of guide processing. The guide processing can be started at any time. The guide processing can be started, for example, when a predetermined application in the surgery assistance device 10 is started.

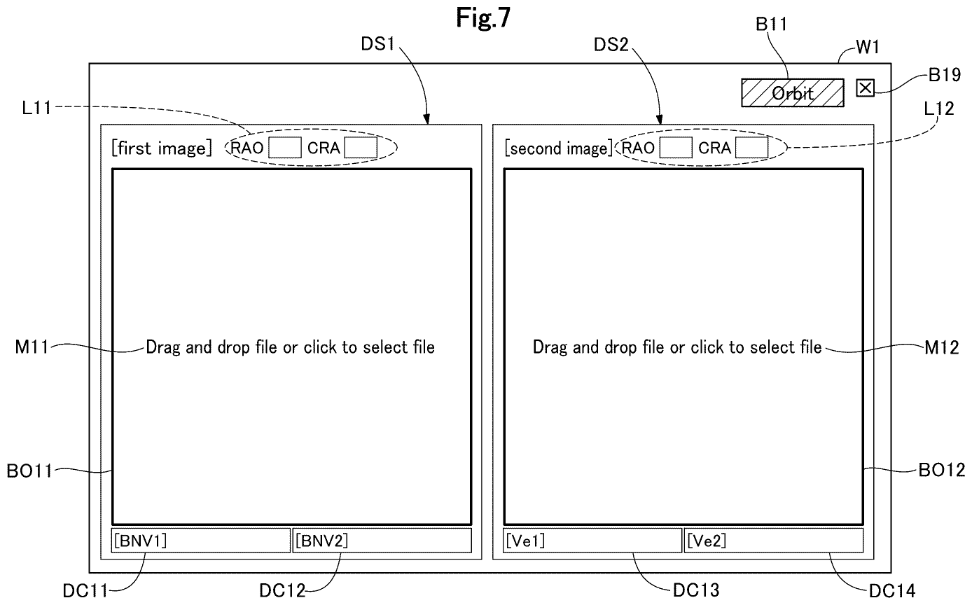

In step S10, the calculation unit 11 of the surgery assistance device 10 acquires a first image and a second image. A first screen W1 (see FIG. 7) is a screen for prompting the operator to input the first image and the second image. The “first image” is an image that has been captured with the FPD 21 in an arbitrary placement position. The “second image” is an image that has been captured with the FPD 21 in an arbitrary placement position that is different to the first position. The first image and the second image both include the target blood vessel. In the first image and the second image, the placement position of the FPD 21 is different. As a result, the target blood vessel included in the first image and the target blood vessel included in the second image show an image of the target blood vessel when viewed from different directions. The placement position of the FPD 21 when the first image is acquired is also referred to as a “first position”. The placement position of the FPD 21 when the second image is acquired is also referred to as a “second position”. In step S10 of the guide processing, the calculation unit 11 displays the first screen W1 on the display device 30 in order to acquire the first image and the second image.

FIG. 7 is a diagram showing an example of the first screen W1 in an initial state. The first screen W1 includes a first image specification portion DS1, a second image specification portion DS2, a trajectory display button B11, and a cancel button B19. In the initial state, the trajectory display button B11 is grayed out. The cancel button B19 is a button for closing the first screen W1.

The first image specification portion DS1 is a section used by the operator to specify the first image. The first image specification portion DS1 includes a first image acquisition FPD position L11, a message M11, a first input field BO11, a display field DC11, and a display field DC12. The message M11 is placed at the center of the first input field BO11, and displays, for example, “Drag and drop file or click to select file”. The second image specification portion DS2 is a section used by the operator to specify the second image. The second image specification portion DS2 includes a second image acquisition FPD position L12, a message M12, a second input field BO12, a display field DC13, and a display field DC14. The message M12 is placed at the center of the second input field BO12, and displays, for example, “Drag and drop file or click to select file”.

The operator follows the message M11, and drags and drops an input target image from a local folder onto the first input field BO11. The operator may click the first input field BO11 instead of dragging and dropping the image. In a case where the first input field BO11 has been clicked, a file selection window is opened for specifying the input target image. The input target image is a still image or a moving image captured in advance by the FPD 21. In the case of a moving image, the operator determines the scene to be captured while referring to the moving image that is automatically played in the first input field BO11. The operator selects, as the capture scene, a scene in which the target blood vessel is clearly visible. Then, the operator follows the message M12, and drags and drops an input target image from a local folder onto the second input field BO12, or specifying an input target image using a selection window. The details are the same as the input to the first input field BO11.

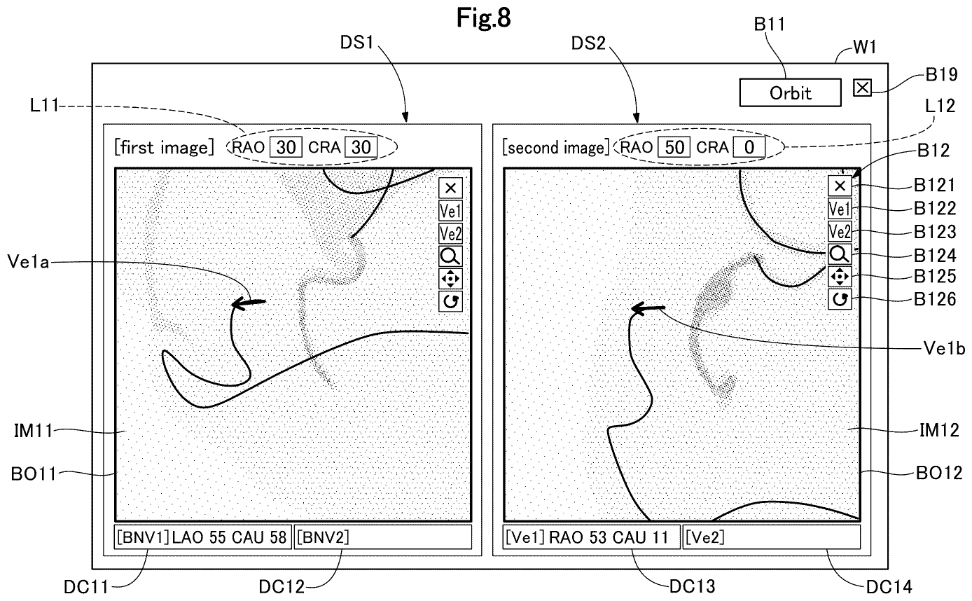

FIG. 8 is an explanatory diagram of an acquisition method of the target blood vessel using the first screen W1. The first input field BO11 shown in FIG. 8 displays the image that has been specified as the first image. Of the first image specification portion DS1, the first image acquisition FPD position L11 is the placement position of the FPD 21 when the first image was captured. In a case where the image that has been specified in the first input field BO11 includes position information of the FPD at the time of image capture, the position information of the FPD that is stored in association with the image is automatically displayed in the first image acquisition FPD position L11. In a case where the image that has been specified in the first input field BO11 does not include position information of the FPD at the time of image capture, the operator inputs the first image acquisition FPD position L11. The second input field BO12 displays the image that has been specified as the second image. Of the second image specification portion DS2, the second image acquisition FPD position L12 is the placement position of the FPD 21 when the second image was captured. The second image acquisition FPD position L12, in a similar manner to the first image, is automatically displayed, or is manually input by the operator. As shown in FIG. 8, the first image and the second image include an image of the heart 91, an image of a cardiac blood vessel, an image of the medical device that has been inserted into the cardiac blood vessel, an image of a contrast medium, and the like.

Image operation buttons B12 are displayed superimposed on the upper right of each of the first image and the second image. For convenience of the illustration, reference signs are displayed in FIG. 8 only for the image operation buttons B12 that are superimposed on the second image. The image operation buttons B12 include, in order from the top, a cancel button B121, a first vector drawing button B122, a second vector drawing button B123, a magnification button B124, a range movement button B125, and a redo button B126. The cancel button B121 is a button for discarding the selected image, and returning the first input field BO11 or the second input field BO12 to the state shown in FIG. 7. The first vector drawing button B122 is a button for the operator to draw a line segment that specifies a target blood vessel. The second vector drawing button B123 is a button for the operator to draw a line segment that specifies a target blood vessel. The target blood vessel specified by the first vector drawing button B122 and the target blood vessel specified by the second vector drawing button B123 are different blood vessels. That is, the second vector drawing button B123 is used in a case where a second target blood vessel is specified. The second vector drawing button B123 can be omitted. The magnification button B124 is a button for magnifying the image that is being displayed in the first input field BO11 or the second input field BO12. The range movement button B125 is a button for moving the center of the image that is being displayed in the first input field BO11 or the second input field BO12. The redo button B126 is a button for canceling the previous operation.

The operator, after pressing the first vector drawing button B122 of the first input field BO11, traces the target blood vessel appearing in the image displayed in the first input field BO11. Then, a first vector Ve1a representing the target blood vessel is displayed on the image in the first input field BO11. The display field DC11 displays the position of the FPD 21 when the first image acquisition FPD position L11 is rotated by 90° around the first vector Ve1a. Then, the operator, after pressing the first vector drawing button B122 of the second input field BO12, traces the target blood vessel appearing in the image displayed in the second input field BO12. For example, the operator can trace the target blood vessel using a mouse cursor. Then, a first vector Ve1b representing the target blood vessel is displayed on the image in the second input field BO12. The display field DC13 displays “a first vector Ve1 representing the vector of the blood vessel axis of the target blood vessel in the three-dimensional space in which the FPD 21 has been placed”. The first vector Ve1 in the display panel DC13 is calculated from the first vector Ve1a and the first vector Ve1b. The first vector Ve1 in the display panel DC13 is the calculation result of step S14 described below. For example, the example of FIG. 8 shows that the first vector Ve1b representing the target blood vessel is equivalent to an arrow from the origin (0, 0, 0) of the three-dimensional space in which the FPD 21 is placed toward the direction RAO 53, CAU 11. The blood vessel represented by the first vector Ve1a and the blood vessel represented by the first vector Ve1b are the same blood vessel. As shown in FIG. 8, after finishing the drawing of the first vectors Ve1a and Ve1b, the trajectory display button B11 is no longer grayed out.

In a case where the operator draws a second vector using the second vector drawing button B123, different arrows to the first vectors Ve1a and Ve1b are displayed in the first input field BO11 and the second input field BO12. The display field DC12 displays the position of the FPD 21 when the first image acquisition FPD position L11 is rotated by 90° around the second vector. In the display panel DC14, the second vector in the three-dimensional space in which the FPD 21 is placed is displayed in a representation that shows the placement position of the FPD 21. The drawing of the second vector is optional and will not be described further.

In step S10 of FIG. 6, the calculation unit 11 acquires the first image and the second image that have been specified on the first screen W1. At this time, the calculation unit 11 acquires both a “first position”, which is the placement position of the FPD 21 when the first image was captured, and a “second position”, which is the placement position of the FPD 21 when the second image was captured. The first position is the value displayed in the first image acquisition FPD position L11. The second position is the value displayed in the second image acquisition FPD position L12. The calculation unit 11, in step S12, acquires the first vector Ve1a and the first vector Ve1b that have been specified on the first screen W1. At this time, the calculation unit 11 acquires both the angles of the first vector Ve1a and the angles of the first vector Ve1b. The angles of the first vector Ve1a are the values displayed in the display panel DC11. The angles of the first vector Ve1b are the values displayed in the display panel DC13.

In step S14, the calculation unit 11 calculates the blood vessel axis of the target blood vessel using the information acquired in steps S10 to S12. In other words, the calculation unit 11 calculates the first vector Ve1 representing the vector of the blood vessel axis of the target blood vessel in the three-dimensional space in which the FPD 21 has been placed. The first vector Ve1 is calculated from the first vectors Ve1a and Ve1b in FIG. 8 that have been drawn by the operator. The details of the method of calculating the vector of a blood vessel axis in a three-dimensional space in which an FPD has been placed using first and second images that have been captured at first and second positions, which are different positions from each other, is disclosed in International Application PCT/JP2021/034980. In International Application PCT/JP2021/034980, a blood vessel presence plane H2 viewed from a first position, and a blood vessel presence plane S viewed from a second position are used to define a straight line on which the H2 plane and the S plane intersect, and then a “blood vessel axis vector”, being the straight line, is calculated. In the example of the present embodiment, for example, the plane on which the first vector Ve1a shown by the first image exists corresponds to the H2 plane, and the first vector Ve1b shown by the second image exists corresponds to the S plane. In this state, the calculation unit 11 calculates the first vector Ve1, which is the vector of the blood vessel axis of the target blood vessel.

In step S20, the two-dimensional image generation unit 12 and the three-dimensional image generation unit 13 of the surgery assistance device 10 detect the pressing of the trajectory display button B11 on the first screen W1. In step S22, the two-dimensional image generation unit 12 generates a two-dimensional image. The “two-dimensional image” is an image showing the placement position of the FPD 21 in two-dimensional coordinates. In step S22, the three-dimensional image generation unit 13 generates a three-dimensional image. The “three-dimensional image” is an image showing the placement position of the FPD 21 in three-dimensional coordinates. In step S24, the composite image generation unit 14 of the surgery assistance device 10 generates a composite image in which the two-dimensional image and the three-dimensional image generated in step S22 have been combined. The “combining” performed by the composite image generation unit 14 refers to forming a single image by arranging the two-dimensional image and the three-dimensional image side-by-side on the left and right. In step S24, the composite image generation unit 14 outputs the generated composite image to the display device 30. In other words, the composite image generation unit 14 displays the composite image on the display device 30.

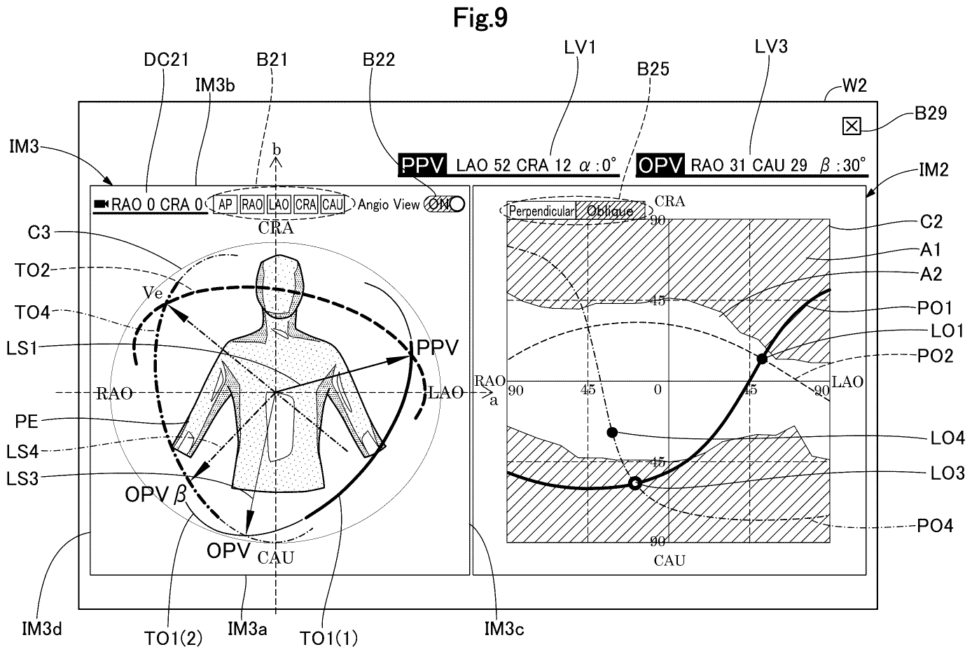

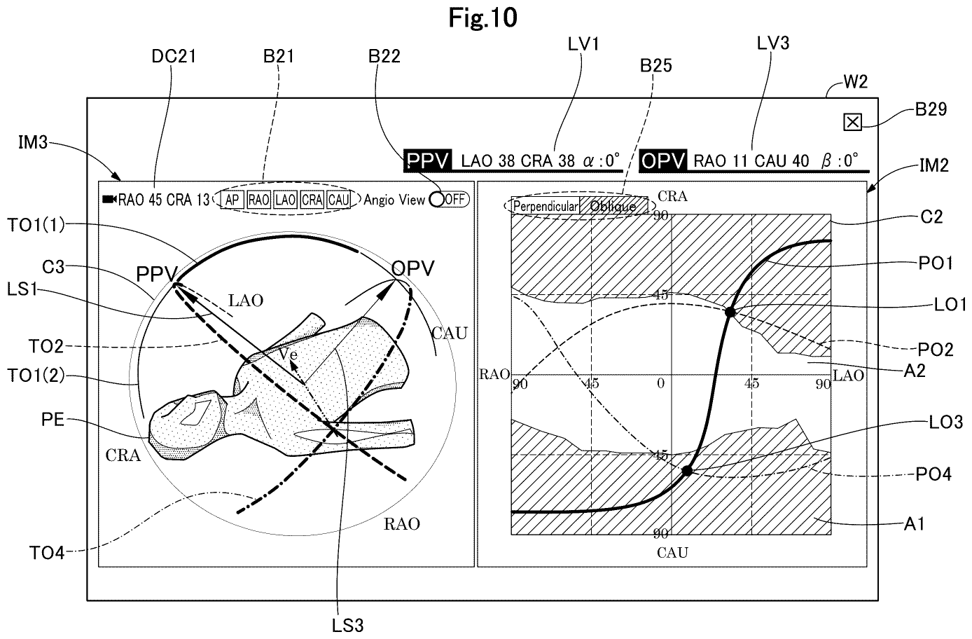

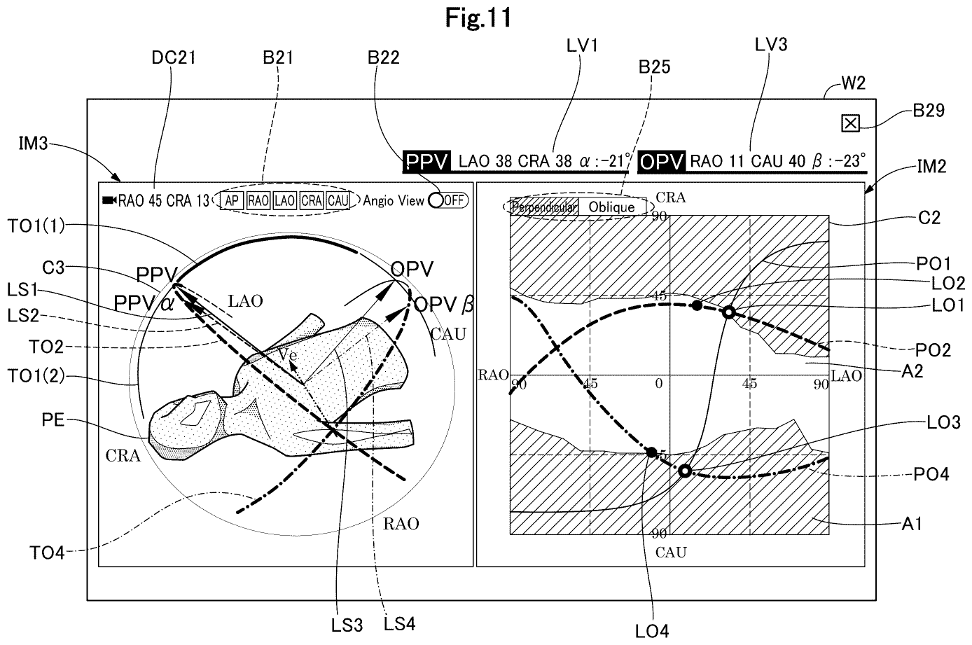

FIG. 9 is a diagram illustrating an example of a composite image. FIGS. 10 and 11 are diagrams illustrating other examples of a composite image. Hereinafter, the screen for displaying the composite image is also referred to as a “second screen W2”. The second screen W2 includes a two-dimensional image IM2, a three-dimensional image IM3, a vertical position LV1, a normal position LV3, and a cancel button B29.

The two-dimensional image IM2 is the two-dimensional image generated by the two-dimensional image generation unit 12. The two-dimensional image IM2 includes b1 to b9 below.

(b1) Two-dimensional coordinates C2: Coordinates representing the three-dimensional space in which the FPD 21 is placed using a Mercator projection. “0” represents the origin of the two-dimensional space. The origin of the two-dimensional coordinates C2 is RAO=0, LAO=0, CRA=0, CAU=0. The two-dimensional coordinates C2 include a square grid and numbers representing the magnitude of the angles θ1 and θ2 from the origin of the two-dimensional coordinates C2 toward each of the directions LAO, RAO, CRA, and CAU described in FIGS. 2 to 5.

(b2) Non-movable range A1, movable range A2: The non-movable range A1 represents a range in which the FPD 21 cannot be moved. A hatching is applied to the non-movable range A1. The non-movable range A1 may be filled in with a dark color. The movable range A2 represents a range in which the FPD 21 can be moved. The movable range A2 is filled in with a white color. The movable range A2 may be filled in with a lighter color than the non-movable range A1. The movable range of the FPD 21 is determined in advance, and is stored in the storage unit of the surgery assistance device 10.

(b3) First planar trajectory PO1: The first planar trajectory PO1 is a trajectory connecting points at which the placement position of the FPD 21 is positioned in a first direction, which is perpendicular to the extending direction of the blood vessel axis Ve. The blood vessel axis Ve is the first vector Ve1 obtained in step S14, which represents the vector of the blood vessel axis of the target blood vessel. The calculation unit 11 can calculate the first planar trajectory PO1 using the blood vessel axis Ve. The first planar trajectory PO1 can also be referred to as a first planar range PO1. In the illustrated example, the first planar trajectory PO1 is represented by a bold and solid curved line.

(b4) First position LO1: The first position LO1 is an arbitrary placement position of the FPD 21 on the first planar trajectory PO1. The first position LO1 can be said to be an arbitrary point on the first planar trajectory PO1. In the example of FIGS. 9 and 10, the first position LO1 is represented by a black circle. In the example of FIG. 11, the first position LO1 is represented by a hollow circle. In a case where the first position LO1 is a hollow circle, this indicates that a second position LO2 based on the first position LO1 exists (see FIG. 11).

(b5) Second planar trajectory PO2: The second planar trajectory PO2 is a trajectory connecting points at which the placement position of the FPD 21 is positioned in the second direction, which is on a first virtual plane that passes through the extending direction of the blood vessel axis Ve, and a virtual line segment that extends from the origin of the three-dimensional space in which the FPD 21 is placed, to the first position LO1. The second planar trajectory PO2 can also be referred to as a second planar range PO2. In the illustrated example, the second planar trajectory PO2 is represented by a dashed line.

(b6) Second position LO2: The second position LO2 is an arbitrary placement position of the FPD 21 on the second planar trajectory PO2. The second position LO2 can be said to be an arbitrary point on the second planar trajectory PO2. The second position LO2 represents the placement position of the FPD 21 based on the first position LO1 when the C-arm 23 is tilted in a state where the FPD 21 is positioned on the first virtual plane. The movement amount of the second position LO2 based on the first position LO1 is represented by “α”. In the example of FIGS. 9 and 10, because α=0° at the vertical position LV1, a point representing the second position LO2 is not drawn. In the example of FIG. 11, the second position LO2 is represented by a black circle.

(b7) Third position LO3: The third position LO3 is a position that is separated from the first position LO1 on the first planar trajectory PO1. The third position LO3 can be specified as follows. First, the first virtual plane is defined that passes through the extending direction of the blood vessel axis Ve, and a virtual line segment that extends from the origin of the three-dimensional space in which the FPD 21 is placed, to the first position LO1. The third position LO3 is a point on a second virtual plane, which is obtained by rotating the first virtual plane by 90° around the extending direction of the blood vessel axis Ve. The third position LO3 can be said to be a placement position of the FPD 21 that is on the first planar trajectory PO1, and separated by 90° with respect to the first position LO1. In the example of FIGS. 9 and 11, the third position LO3 is represented by a hollow circle. In the example of FIG. 10, the third position LO3 is represented by a black circle. In a case where the third position LO3 is a hollow circle, this indicates that a fourth position LO4 based on the third position LO3 exists. As is clear from FIGS. 9 and 10, the third position LO3 changes in response to a change in the position of the first position LO1. In other words, the third position LO3 moves so as to follow the first position LO1.

(b8) Fourth planar trajectory PO4: The fourth planar trajectory PO4 is a trajectory connecting points at which the placement position of the FPD 21 is positioned in a fourth direction on the second virtual plane. The second virtual plane is the same as the second virtual plane described in relation to the third position LO3. The fourth planar trajectory PO4 can also be referred to as a fourth planar range PO4. In the illustrated example, the fourth planar trajectory PO4 is represented by a dash-dotted line.

(b9) Fourth position LO4: The fourth position LO4 is an arbitrary placement position of the FPD 21 on the fourth planar trajectory PO4. The fourth position LO4 can be said to be an arbitrary point on the fourth planar trajectory PO4. The fourth position LO4 represents the placement position of the FPD 21 based on the third position LO3 when the C-arm 23 is tilted in a state where the FPD 21 is positioned on the second virtual plane. The movement amount of the fourth position LO4 based on the third position LO3 is represented by “β”. In the example of FIG. 10, because β=0° in the normal position LV3, a point representing the fourth position LO4 is not drawn. In the example of FIGS. 9 and 11, the fourth position LO4 is represented by a black circle.

The other elements included in the two-dimensional image IM2 will be described. The mode specification button B25 is a button for switching between a Perpendicular mode and an Oblique mode. In the case of the Perpendicular mode, the first planar trajectory PO1 is shown with bold lines, and the operator is capable of moving the first position LO1 and the third position LO3 using the mouse. For example, in a case where the first position LO1 is moved, the operator moves the first position LO1 on the first planar trajectory PO1 by moving the mouse cursor in an arbitrary direction of the first planar trajectory PO1 in a state where the point representing the original first position LO1 is selected. In the case of the oblique mode, the second planar trajectory PO2 and the fourth planar trajectory PO4 are shown with bold lines, and the operator is capable of moving the second position LO2 and the fourth position LO4 using the mouse. In the Oblique mode, the first position LO1 and the third position LO3 are fixed.

The three-dimensional image IM3 is a three-dimensional image generated by the three-dimensional image generation unit 13. The three-dimensional image IM3 includes c1 to c9 below.

(c1) Three-dimensional coordinates C3: Coordinates representing the three-dimensional space in which the FPD 21 is placed, and includes a virtual spherical model having a predetermined radius. The spherical model has the origin (center) at the center of rotation of the C-arm 23, and is a spherical model having a radius such that the FPD 21 fixed to the C-arm 23 moves along the surface of the sphere. The spherical model may, for example, be divided into a grid having a predetermined size such as on a globe.

(c2) Patient image PE, blood vessel axis Ve: A three-dimensional model image of the human body 90. The head portion of the human body 90 is positioned in the CRA direction. The blood vessel axis Ve is displayed superimposed on the patient image PE. The blood vessel axis Ve is an arrow passing through the origin of the three-dimensional coordinates C3, and extending in the direction calculated in step S14 of FIG. 6. That is, the origin of the three-dimensional coordinates C3 is the center of rotation of the C-arm 23 (in other words, the origin of the three-dimensional space in which the FPD 21 is placed), and can be said to be an arbitrary point on the target blood vessel. In the illustrated example, the blood vessel axis Ve is represented by a two-dot chain arrow.

(c3) First three-dimensional trajectory TO1: The first three-dimensional trajectory TO1 is a trajectory representing the first planar trajectory PO1 on the spherical model in the three-dimensional coordinates C3. The first three-dimensional trajectory TO1 can also be referred to as a first three-dimensional range TO1. The first three-dimensional trajectory TO1 represents the first planar trajectory PO1 as a circumference of a circle having a radius of a predetermined length from the origin of the three-dimensional coordinates C3. As illustrated by TO1(1) in FIGS. 9 to 11, the section of the first three-dimensional trajectory TO1 that overlaps the movable range A2 of the FPD 21 is illustrated with a bold solid line. As illustrated by TO1(2) in FIGS. 9 to 11, the section of the first three-dimensional trajectory TO1 that overlaps the non-movable range A1 of the FPD 21 is represented by a thin solid line.

(c4) First line segment LS1: The first line segment LS1 is an arrow showing the first position LO1 on the first planar trajectory PO1 as a line segment extending from the origin of the three-dimensional coordinates C3 to the first position LO1. That is, the first line segment LS1 and the first position LO1 correspond to each other. In the illustrated example, the first line segment LS1 is represented by a bold solid arrow. The front end side of the arrow of the first line segment LS1 is shown with a character string “PPV”.

(c5) Second three-dimensional trajectory TO2: The second three-dimensional trajectory TO2 is a trajectory representing the second planar trajectory PO2 on the spherical model in the three-dimensional coordinates C3. The second three-dimensional trajectory TO2 can also be referred to as a second three-dimensional range TO2. The second three-dimensional trajectory TO2 represents the second planar trajectory PO2 as a circumference of a circle including the first virtual plane, and having a radius of a predetermined length from the origin of the three-dimensional coordinates C3. On the second three-dimensional trajectory TO2, the section overlapping the movable range A2 of the FPD 21 is illustrated with a bold dashed line, and the section overlapping the non-movable range A1 of the FPD 21 is represented by a thin dashed line.

(c6) Second line segment LS2: The second line segment LS2 is an arrow showing the second position LO2 on the second planar trajectory PO2 as a line segment extending from the origin of the three-dimensional coordinates C3 to the second position LO2. That is, the second line segment LS2 and the second position LO2 correspond to each other. In the example of FIGS. 9 and 10, because α=0°, the second line segment LS2 is not drawn. In the example of FIG. 11, the second line segment LS2 is represented by a dashed arrow. The front end side of the arrow of the second line segment LS2 is shown with a character string “PPVα”.

(c7) Third line segment LS3: The third line segment LS3 is an arrow showing the third position LO3 on the first planar trajectory PO1 as a line segment extending from the origin of the three-dimensional coordinates C3 to the third position LO3. That is, the third line segment LS3 and the third position LO3 correspond to each other. In the illustrated example, the third line segment LS3 is represented by a thin solid arrow. The front end side of the arrow of the third line segment LS3 is shown with a character string “OPV”.

(c8) Fourth three-dimensional trajectory TO4: The fourth three-dimensional trajectory TO4 is a trajectory representing the fourth planar trajectory PO4 on the spherical model in the three-dimensional coordinates C3. The fourth three-dimensional trajectory TO4 can also be referred to as a fourth three-dimensional range TO4. The fourth three-dimensional trajectory TO4 represents the fourth planar trajectory PO4 as a circumference of a circle including the second virtual plane, and having a radius of a predetermined length from the origin of the three-dimensional coordinates C3. On the fourth three-dimensional trajectory TO4, the section overlapping the movable range A2 of the FPD 21 is illustrated with a bold dash-dotted line, and the section overlapping the non-movable range A1 of the FPD 21 is represented by a thin dash-dotted line.

(c9) Fourth line segment LS4: The fourth line segment LS4 is an arrow showing the fourth position LO4 on the fourth planar trajectory PO4 as a line segment extending from the origin of the three-dimensional coordinates C3 to the fourth position LO4. That is, the fourth line segment LS4 and the fourth position LO4 correspond to each other. In the example of FIG. 10, because β=0°, the fourth line segment LS4 is not drawn. In the example of FIGS. 9 and 11, the fourth line segment LS4 is represented by a dash-dotted arrow. The front end side of the arrow of the second line segment LS2 is shown with a character string “OPVβ”.

The other elements included in the three-dimensional image IM3 will be described. The display panel DC21 displays the viewpoint position that is being currently displayed in three-dimensional coordinates C3. For example, in the example of FIG. 9, assuming that the FPD 21 moves on the surface of a virtual sphere of radius 1 in a three-dimensional space, the viewpoint is from (x, y, z)=(0, 1, 0), and therefore, RAO0 and CRA0 are displayed.

The viewpoint change buttons B21 are buttons for changing the viewpoint that is displayed in the three-dimensional coordinates C3. By pressing the AP button, the viewpoint becomes a view of the three-dimensional coordinates C3 and the patient image PE from the FPD 21 placed at LAO=0, RAO=0, CRA=0, and CAU=0. By pressing the RAO button, the viewpoint becomes a view of the three-dimensional coordinates C3 and the patient image PE from the RAO direction. “From the RAO direction” may be a view where RAO=90°, or may be approximately RAO=70° to match the viewpoint from which the operator views the patient. The same also applies to the LAO, CRA, and CAU directions. By pressing the LAO button, the viewpoint becomes a view of the three-dimensional coordinates C3 and the patient image PE from the LAO direction. By pressing the CRA button, the viewpoint becomes a view of the three-dimensional coordinates C3 and the patient image PE from the CRA direction. By pressing the CAU button, the viewpoint becomes a view of the three-dimensional coordinates C3 and the patient image PE from the CAU direction. In addition to changing the viewpoint by pressing these buttons, the operator is capable of changing the viewpoint by moving the mouse cursor in a state where an arbitrary point in the three-dimensional coordinates C3 is selected, and then rotating the three-dimensional coordinates C3 and the patient image PE in an arbitrary direction. The first to fourth three-dimensional trajectories TO1 to TO4 and the first to fourth line segments LS1 to LS4 also rotate with the rotation of the three-dimensional coordinates C3 and the patient image PE.

The head portion position fixing button B22 can be switched ON/OFF. In a case where the head portion position fixing button B22 is in the ON state, the head portion of the patient image PE becomes fixed. As shown by the dashed arrow in FIG. 9, in the three-dimensional image IM3, a-b coordinates, which are two-dimensional coordinates passing through the origin of the three-dimensional coordinates C3, are virtually defined. As shown in FIG. 9, the three-dimensional image IM3 has a bottom side IM3a, a top side IM3b, a right side IM3c, and a left side IM3d, and has a rectangular shape in which all of the internal angles are 90°. A rectangular shape conceptually includes a square shape, in which the lengths of the four sides Im3a to IM3d are equal. The a-axis of the a-b coordinates is parallel to the bottom side IM3a of the three-dimensional image IM3. The positive a-axis direction is the left hand direction of the patient image PE laying down, which is displayed in the three-dimensional image IM3 when the AP button of the viewpoint change buttons B21 is pressed. In the example of FIG. 9, the positive a-axis direction is the direction of the right side IM3c of the three-dimensional image IM3. The b-axis of the a-b coordinates is perpendicular to the a-axis. The positive b-axis direction is the direction of the head portion of the patient image PE laying down, which is displayed in the three-dimensional image IM3 when the AP button of the viewpoint change buttons B21 is pressed. In the example of FIG. 9, the positive b-axis direction is the direction of the top side IM3b of the three-dimensional image IM3.

In a case where the operator performs an operation that rotates the patient image PE when the head portion position fixing button B22 is in the ON state, the patient image PE is rotated in a state where the head portion is fixed on the b axis with b≥0. In other words, when the head portion position fixing button B22 is in the ON state, the operator is capable of rotating the patient image PE and the three-dimensional coordinates C3 on the a-b plane, and further, is capable of performing three-dimensional rotation in a direction passing through the a-b plane. In a case where the head portion position fixing button B22 is in the OFF state, as shown in FIGS. 10 and 11, the head portion of the patient image PE becomes movable in an arbitrary direction according to an operation by the operator. In the present embodiment, the “crown portion” of the head in the patient image PE is fixed, and the section below the crown portion rotates. The entire head portion in the patient image PE may be fixed, and the section below the head may be rotatable.

In the Perpendicular mode, the operator moves the first position LO1 displayed on the first planar trajectory PO1 while referring to the first planar trajectory PO1 and the movable range A2 displayed in the two-dimensional image IM2. Then, in the three-dimensional image IM3, the front end position of the arrow of the first line segment LS1 moves so as to follow the change in the first position LO1. That is, the first position LO1 and the first line segment LS1 are linked to each other. As a result, by referring to the three-dimensional image IM3, the operator can immediately grasp where the first position LO1 that has been specified in the two-dimensional image IM2 is positioned in the three-dimensional coordinates C3 corresponding to the actual three-dimensional space (the three-dimensional space in which the FPD 21 is placed).

In a case where the first position LO1 is moved, the third position LO3 moves in the two-dimensional image IM2, and the front end position of the arrow of the third line segment LS3 moves in the three-dimensional image IM3 so as to follow the movement of the first position LO1. That is, the third position LO3 and the third line segment LS3 are linked to each other. As a result, the operator may move the third position LO3 instead of moving the first position LO1. In a case where the third position LO3 is moved, the first position LO1 moves in the two-dimensional image IM2, and the front end position of the arrow of the first line segment LS1 moves in the three-dimensional image IM3 so as to follow the movement of the third position LO3.

In the Oblique mode, the operator moves the second position LO2 displayed on the second planar trajectory PO2 while referring to the second planar trajectory PO2 and the movable range A2 displayed in the two-dimensional image IM2. At this time, the first position LO1 changes from a black circle to a hollow circle. Then, in the three-dimensional image IM3, the front end position of the arrow of the second line segment LS2 moves so as to follow the change in the second position LO2. That is, the second position LO2 and the second line segment LS2 are linked to each other. As a result, by referring to the three-dimensional image IM3, the operator can immediately grasp where the second position LO2 that has been specified in the two-dimensional image IM2 is positioned in the three-dimensional coordinates C3 corresponding to the actual three-dimensional space.

In the Oblique mode, the operator moves the fourth position LO4 displayed on the fourth planar trajectory PO4 while referring to the fourth planar trajectory PO4 and the movable range A2 displayed in the two-dimensional image IM2. At this time, the third position LO3 changes from a black circle to a hollow circle. Then, in the three-dimensional image IM3, the front end position of the arrow of the fourth line segment LS4 moves so as to follow the change in the fourth position LO4. That is, the fourth position LO4 and the fourth line segment LS4 are linked to each other. As a result, by referring to the three-dimensional image IM3, the operator can immediately grasp where the fourth position LO4 that has been specified in the two-dimensional image IM2 is positioned in the three-dimensional coordinates C3 corresponding to the actual three-dimensional space.

The vertical position LV1 is a character string indicating the placement position of the FPD 21 that is specified by either one of the first position LO1 and the second position LO2. In a case where the first position LO1 is selected, the vertical position LV1 displays the angle information “LAO 52 CRA 12” of the first position LO1, and 0° as the α value. In a case where the second position LO2 is selected, the vertical position LV1 displays the angle information of the second position LO2, and a “value indicating how far away the second position LO2 is from the first position LO1” as the α value. The normal position LV3 is a character string indicating the placement position of the FPD 21 that is specified by either one of the third position LO3 and the fourth position LO4. In a case where the third position LO3 is selected, the normal position LV3 displays the angle information “LAO 31 CAU 29” of the third position LO3, and 0° as the β value. In a case where the fourth position LO4 is selected, the normal position LV3 displays the angle information of the fourth position LO4, and a “value indicating how far away the fourth position LO4 is from the third position LO3” as the β value. The cancel button B29 is a button for closing the second screen W2.

The description will be continued returning to FIG. 6. In step S30, the two-dimensional image generation unit 12 detects a change in the first to fourth positions LO1 to LO4 in the two-dimensional image IM2. In a case where a position has changed (step S30: YES), in step S32, the two-dimensional image generation unit 12 generates a two-dimensional image IM2 reflecting the position after the change. In step S32, the three-dimensional image generation unit 13 generates a three-dimensional image IM3 reflecting the position after the change. In step S34, the composite image generation unit 14 of the surgery assistance device 10 generates a composite image, that is, the second screen W2, in which the two-dimensional image IM2 and the three-dimensional image IM3 generated in step S32 are combined, and then outputs the composite image. In steps S30 to S34, the second screen W2 is updated with the change in the position described above. In step S40, the two-dimensional image generation unit 12 determines whether or not a termination condition has been met. In a case where the termination condition has been met (step S40: YES), the two-dimensional image generation unit 12 ends the processing. The termination condition can be arbitrarily set. The termination condition, for example, can be set as a case where pressing of the cancel button B29 has been detected.

As described above, according to the surgery assistance device 10, the three-dimensional image generation unit 13 generates the three-dimensional image IM3 that represents the placement position of the FPD 21 in the three-dimensional coordinates C3, the three-dimensional image IM3 including the blood vessel axis Ve and the first three-dimensional trajectory TO1. The composite image generation unit 14 generates and outputs the second screen W2 serving as a composite image, in which the two-dimensional image IM2 and the three-dimensional image IM3 have been combined. As a result, the operator can directly grasp the trajectory of the placement position of the FPD 21 in the actual three-dimensional space by referring to not only the two-dimensional image IM2, but also the three-dimensional image IM3. As a result, the operator is capable of accurately and efficiently deriving the optimal placement position of the FPD 21.

Further, according to the surgery assistance device 10, because the two-dimensional image IM2 includes the first position LO1, and the three-dimensional image IM3 includes the first line segment LS1, the operator can easily recognize the placement position of the FPD 21 in the image. As a result, the usability of the surgery assistance device 10 can be improved.

In addition, according to the surgery assistance device 10, because the first position LO1 and the first line segment LS1 are linked to each other, the operator can more easily recognize the placement position of the FPD 21 in the image. As a result, the usability of the surgery assistance device 10 can be further improved.

Also, according to the surgery assistance device 10, the two-dimensional image generation unit 12 generates the two-dimensional image IM2 including the second planar trajectory PO2, and the three-dimensional image generation unit 13 generates the three-dimensional image IM3 including the second three-dimensional trajectory TO2. As a result, the operator is capable of directly grasping the trajectory of the placement position of the FPD 21 with respect to the first position LO1 when the C-arm 23 is tilted in a state where the FPD 21 is positioned on the first virtual plane. As a result, the operator is capable of more accurately and efficiently deriving the optimal placement position of the FPD 21. Further, because the two-dimensional image IM2 includes the second position LO2, and the three-dimensional image IM3 includes the second line segment LS2, the operator can easily recognize the placement position of the FPD 21 in the image. As a result, the usability of the surgery assistance device 10 can be improved. Further, because the second position LO2 and the second line segment LS2 are linked to each other, the operator can more easily recognize the placement position of the FPD 21 in the image. As a result, the usability of the surgery assistance device 10 can be further improved.

Further, according to the surgery assistance device 10, the two-dimensional image IM2 includes the third position LO3, and the three-dimensional image IM3 includes the third line segment LS3. As a result, the operator is capable of directly grasping the placement position of the FPD 21 that is on the first planar trajectory PO1, and separated by 90°with respect to the first position LO1. As a result, the operator is capable of more accurately and efficiently deriving the optimal placement position of the FPD 21. Further, because the third position LO3 and the third line segment LS3 are linked to each other, the operator can more easily recognize the placement position of the FPD 21 in the image. As a result, the usability of the surgery assistance device 10 can be further improved.

In addition, according to the surgery assistance device 10, the two-dimensional image generation unit 12 generates the two-dimensional image IM2 including the fourth planar trajectory PO4, and the three-dimensional image generation unit 13 generates the three-dimensional image IM3 including the fourth three-dimensional trajectory TO4. As a result, the operator is capable of directly grasping the trajectory of the placement position of the FPD 21 with respect to the third position LO3 when the C-arm 23 is tilted in a state where the FPD 21 is positioned on the second virtual plane. As a result, the operator is capable of more accurately and efficiently deriving the optimal placement position of the FPD 21. Further, because the two-dimensional image IM2 includes the fourth position LO4, and the three-dimensional image IM3 includes the fourth line segment LS4, the operator can easily recognize the placement position of the FPD 21 in the image. As a result, the usability of the surgery assistance device 10 can be improved. Further, because the fourth position LO4 and the fourth line segment LS4 are linked to each other, the operator can more easily recognize the placement position of the FPD 21 in the image. As a result, the usability of the surgery assistance device 10 can be further improved.

Also, according to the surgery assistance device 10, the operator is capable of changing the viewpoint of the three-dimensional image IM3 using the viewpoint change buttons B21 or a mouse. As a result, the usability of the surgery assistance device 10 can be further improved. In addition, the operator is capable of using the head portion position fixing button B22 to fix the head portion of the patient image PE in a predetermined direction in the three-dimensional image IM3. As a result, the usability of the surgery assistance device 10 can be further improved.

In addition, according to the surgery assistance device 10, the second screen W2 serving as the composite image includes the vertical position LV1, which corresponds to one of a character string indicating the position of the FPD 21 for placing the FPD 21 in the first position LO1, and a character string indicating the position of the FPD 21 for placing the FPD 21 in the second position LO2. As a result, it becomes easier for the operator to use the character string and specify the position of the FPD 21 with respect to the radiation imaging device 20.

In addition, according to the surgery assistance device 10, the second screen W2 serving as the composite image includes the normal position LV3, which corresponds to one of a character string indicating the position of the FPD 21 for placing the FPD 21 in the third position LO3, and a character string indicating the position of the FPD 21 for placing the FPD 21 in the fourth position LO4. As a result, it becomes easier for the operator to use the character string and specify the position of the FPD 21 with respect to the radiation imaging device 20.

### Second Embodiment

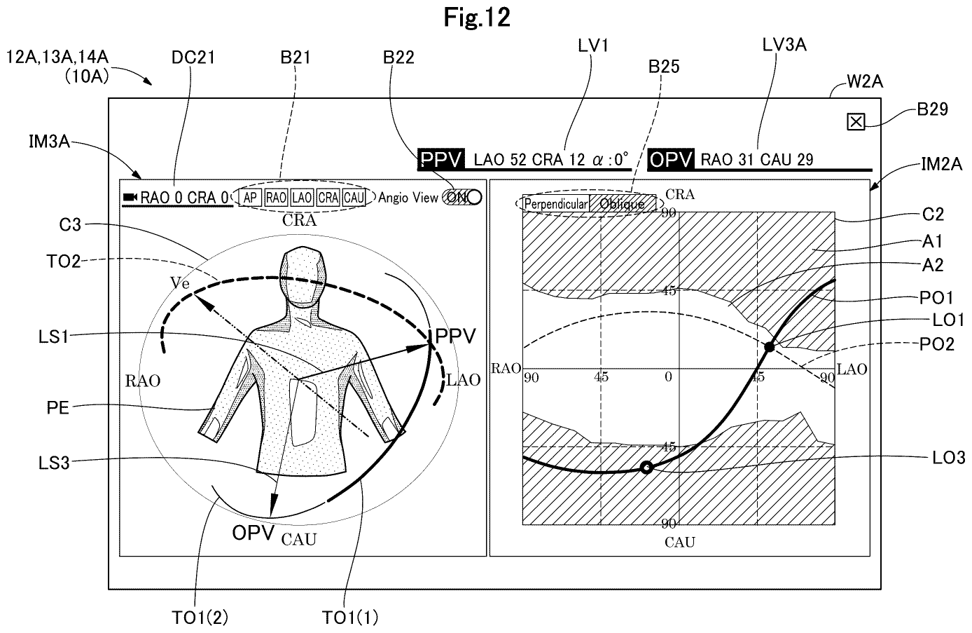

FIG. 12 is a diagram showing an example of a composite image according to a second embodiment. The surgery assistance system 1 according to the second embodiment includes a surgery assistance device 10A instead of the surgery assistance device 10 described in the first embodiment. The surgery assistance device 10A includes a two-dimensional image generation unit 12A instead of the two-dimensional image generation unit 12, a three-dimensional image generation unit 13A instead of the three-dimensional image generation unit 13, and a composite image generation unit 14A instead of the composite image generation unit 14. The two-dimensional image generation unit 12A generates the two-dimensional image IM2A shown in FIG. 12. The three-dimensional image generation unit 13A generates the three-dimensional image IM3A shown in FIG. 12. The composite image generation unit 14A generates and outputs the second screen W2A shown in FIG. 12.

The two-dimensional image IM2A does not include the fourth planar trajectory PO4 and the fourth position LO4 described in the first embodiment. The three-dimensional image IM3A does not include the fourth three-dimensional trajectory TO4 and the fourth line segment LS4 described in the first embodiment. The normal position LV3A does not include the β value of the fourth position LO4.

In this way, various modifications are possible to the form of the two-dimensional image IM2A and the three-dimensional image IM3A. The surgery assistance device 10A according to the second embodiment is capable of achieving the same effects as those of the first embodiment described above.

### Third Embodiment

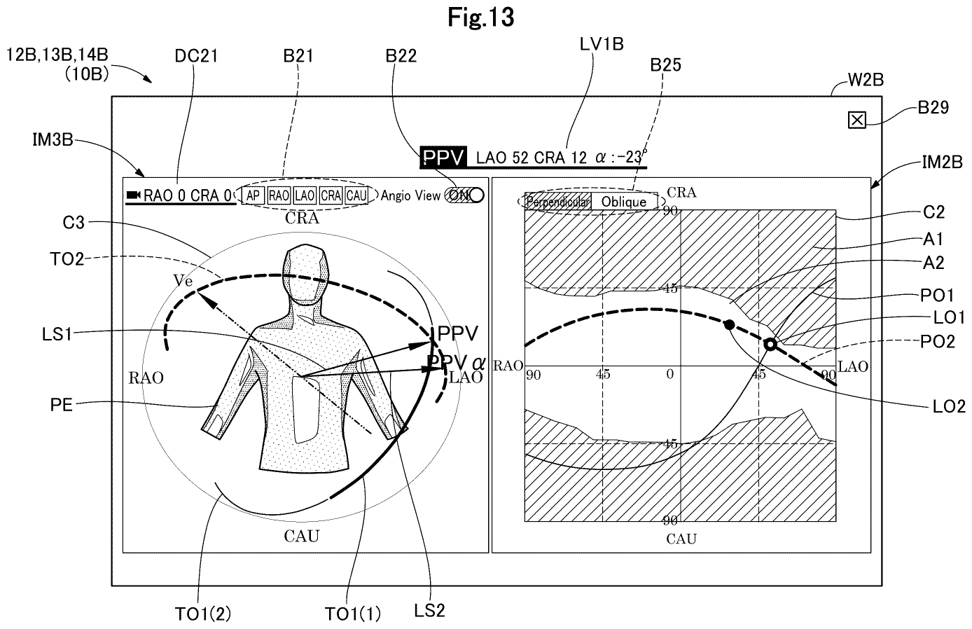

FIG. 13 is a diagram showing an example of a composite image according to a third embodiment. The surgery assistance system 1 according to the third embodiment includes a surgery assistance device 10B instead of the surgery assistance device 10 described in the first embodiment. The surgery assistance device 10B includes a two-dimensional image generation unit 12B instead of the two-dimensional image generation unit 12, a three-dimensional image generation unit 13B instead of the three-dimensional image generation unit 13, and a composite image generation unit 14B instead of the composite image generation unit 14. The two-dimensional image generation unit 12B generates the two-dimensional image IM2B shown in FIG. 13. The three-dimensional image generation unit 13B generates the three-dimensional image IM3B shown in FIG. 13. The composite image generation unit 14B generates and outputs the second screen W2B shown in FIG. 13.

The two-dimensional image IM2B does not include the third position LO3, the fourth planar trajectory PO4, and the fourth position LO4 described in the first embodiment. The three-dimensional image IM3B does not include the third line segment LS3, the fourth three-dimensional trajectory TO4, and the fourth line segment LS4 described in the first embodiment. The second screen W2B does not include the normal position LV3 described in the first embodiment.

In this way, various modifications are possible to the form of the two-dimensional image IM2B and the three-dimensional image IM3B. The surgery assistance device 10B according to the third embodiment is capable of achieving the same effects as those of the first embodiment described above.

### Fourth Embodiment

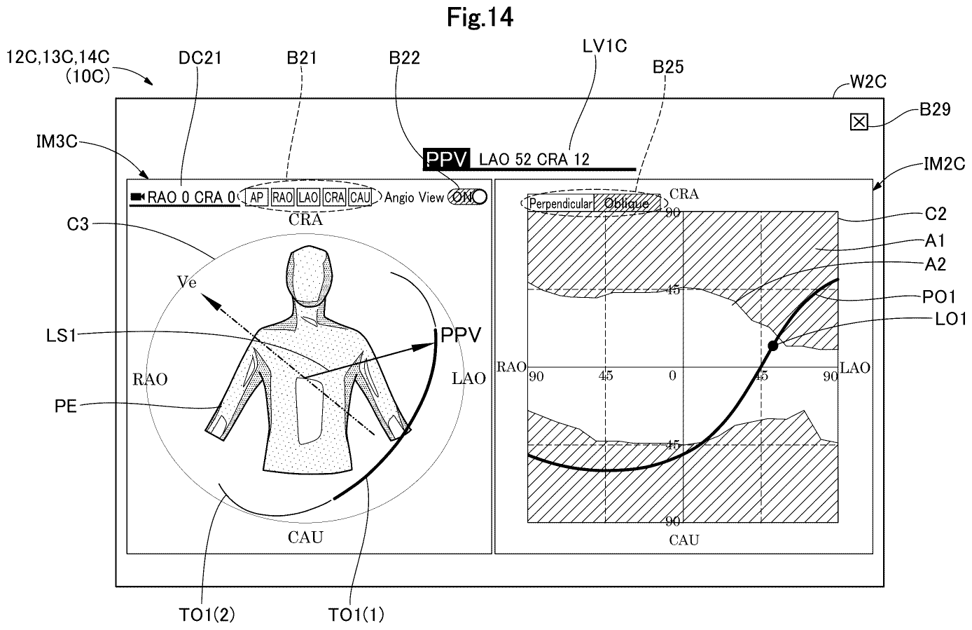

FIG. 14 is a diagram showing an example of a composite image according to a fourth embodiment. The surgery assistance system 1 according to the fourth embodiment includes a surgery assistance device 10C instead of the surgery assistance device 10 described in the first embodiment. The surgery assistance device 10C includes a two-dimensional image generation unit 12C instead of the two-dimensional image generation unit 12, a three-dimensional image generation unit 13C instead of the three-dimensional image generation unit 13, and a composite image generation unit 14C instead of the composite image generation unit 14. The two-dimensional image generation unit 12C generates the two-dimensional image IM2C shown in FIG. 14. The three-dimensional image generation unit 13C generates the three-dimensional image IM3C shown in FIG. 14. The composite image generation unit 14C generates and outputs the second screen W2C shown in FIG. 14.

The two-dimensional image IM2C does not include the second planar trajectory PO2, the second position LO2, the third position LO3, the fourth planar trajectory PO4, and the fourth position LO4 described in the first embodiment. The three-dimensional image IM3C does not include the second three-dimensional trajectory TO2, the second line segment LS2, the third line segment LS3, the fourth three-dimensional trajectory TO4, and the fourth line segment LS4 described in the first embodiment. The vertical position LV1C does not include the α value of the second position LO2. The second screen W2C does not include the normal position LV3 described in the first embodiment.

In this way, various modifications are possible to the form of the two-dimensional image IM2C and the three-dimensional image IM3C. The surgery assistance device 10C according to the fourth embodiment is capable of achieving the same effects as those of the first embodiment described above.

### Fifth Embodiment

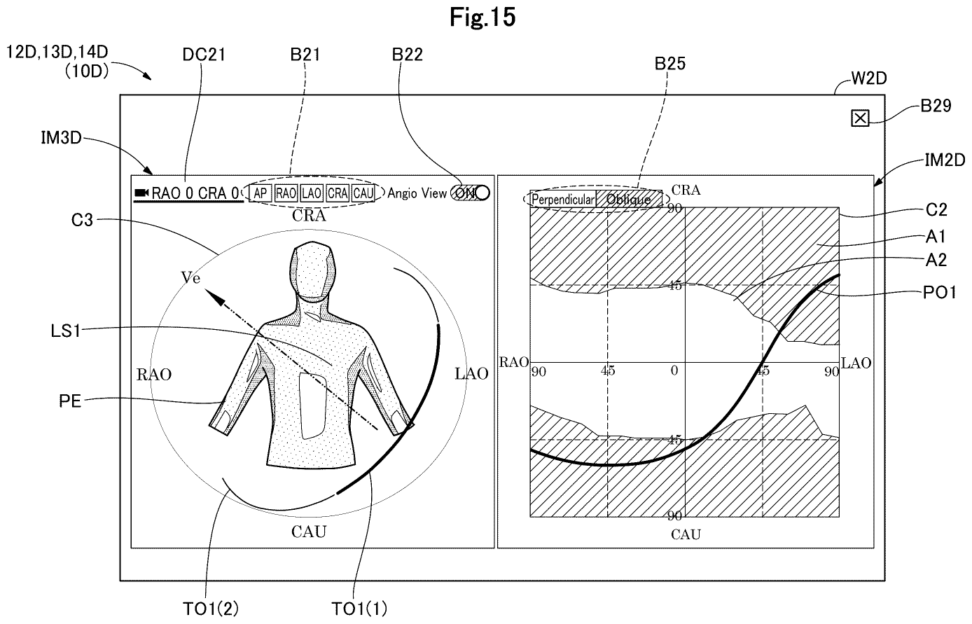

FIG. 15 is a diagram showing an example of a composite image according to a fifth embodiment. The surgery assistance system 1 according to the fifth embodiment includes a surgery assistance device 10D instead of the surgery assistance device 10 described in the first embodiment. The surgery assistance device 10D includes a two-dimensional image generation unit 12D instead of the two-dimensional image generation unit 12, a three-dimensional image generation unit 13D instead of the three-dimensional image generation unit 13, and a composite image generation unit 14D instead of the composite image generation unit 14. The two-dimensional image generation unit 12D generates the two-dimensional image IM2D shown in FIG. 15. The three-dimensional image generation unit 13D generates the three-dimensional image IM3D shown in FIG. 15. The composite image generation unit 14D generates and outputs the second screen W2D shown in FIG. 15.

The two-dimensional image IM2D does not include the first position LO1, the second planar trajectory PO2, the second position LO2, the third position LO3, the fourth planar trajectory PO4, and the fourth position LO4 described in the first embodiment. The three-dimensional image IM3D does not include the first line segment LS1, the second three-dimensional trajectory TO2, the second line segment LS2, the third line segment LS3, the fourth three-dimensional trajectory TO4, and the fourth line segment LS4 described in the first embodiment. The second screen W2D does not include the vertical position LV1 and the normal position LV3 described in the first embodiment.

In this way, various modifications are possible to the form of the two-dimensional image IM2D and the three-dimensional image IM3D. The surgery assistance device 10D according to the fifth embodiment is capable of achieving the same effects as those of the first embodiment described above.

### Sixth Embodiment

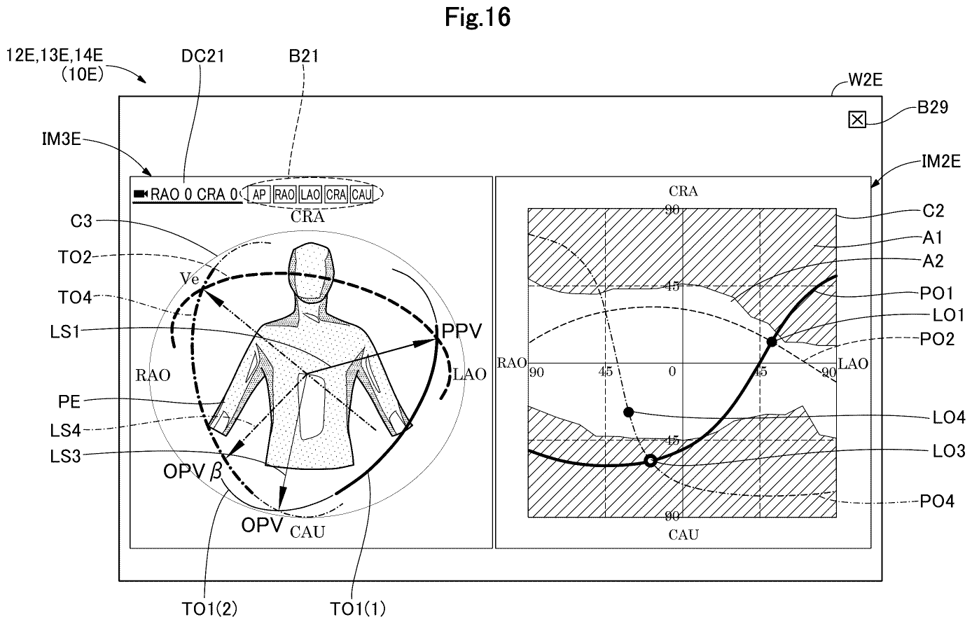

FIG. 16 is a diagram showing an example of a composite image according to a sixth embodiment. The surgery assistance system 1 according to the sixth embodiment includes a surgery assistance device 10E instead of the surgery assistance device 10 described in the first embodiment. The surgery assistance device 10E includes a two-dimensional image generation unit 12E instead of the two-dimensional image generation unit 12, a three-dimensional image generation unit 13E instead of the three-dimensional image generation unit 13, and a composite image generation unit 14E instead of the composite image generation unit 14. The two-dimensional image generation unit 12E generates the two-dimensional image IM2E shown in FIG. 16. The three-dimensional image generation unit 13E generates the three-dimensional image IM3E shown in FIG. 16. The composite image generation unit 14E generates and outputs the second screen W2E shown in FIG. 16.

The two-dimensional image IM2E does not include the mode specification button B25 described in the first embodiment. In the two-dimensional image IM2E, an automatic transition to the Oblique mode occurs as a result of the operator moving the mouse cursor in an arbitrary direction of the second planar trajectory PO2 in a state where the first position LO1 is selected. Similarly, an automatic transition to the Oblique mode occurs as a result of the operator moving the mouse cursor in an arbitrary direction of the fourth planar trajectory PO4 in a state where the third position LO3 is selected. In addition, an automatic transition to the Perpendicular mode occurs as a result of the operator moving the mouse cursor in an arbitrary direction of the first planar trajectory PO1 in a state where either one of the first position LO1 and the third position LO3 are selected. The three-dimensional image IM3E is not provided with the head portion position fixing button B22 described in the first embodiment. The second screen W2E does not include the vertical position LV1 and the normal position LV3 described in the first embodiment.

In this way, various modifications are possible to the form of the two-dimensional image IM2E and the three-dimensional image IM3E, and at least some of the items mentioned above may be omitted. The surgery assistance device 10E according to the sixth embodiment is capable of achieving the same effects as those of the first embodiment described above.

### Seventh Embodiment

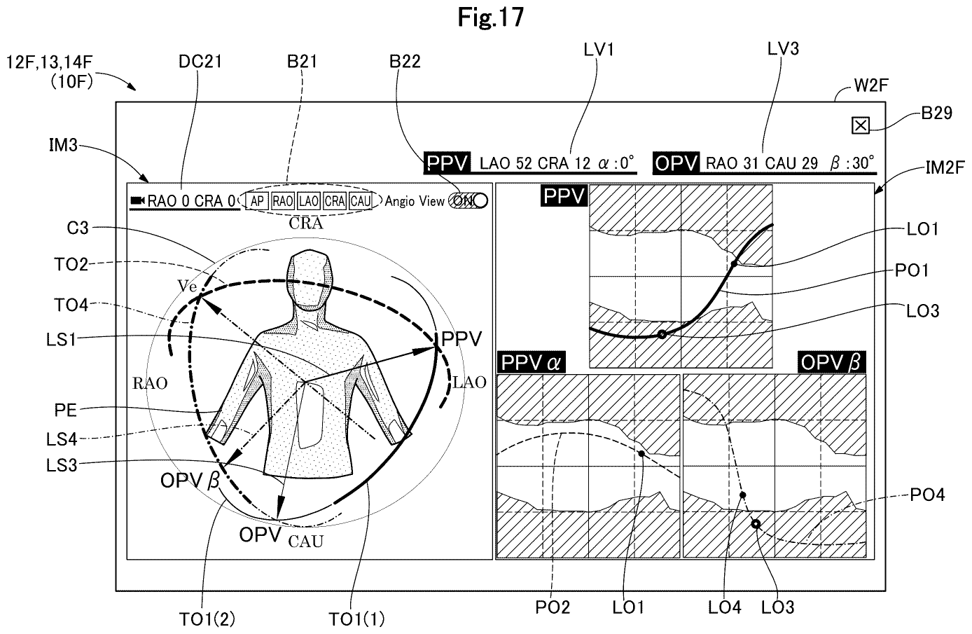

FIG. 17 is a diagram showing an example of a composite image according to a seventh embodiment. The surgery assistance system 1 according to the seventh embodiment includes a surgery assistance device 10F instead of the surgery assistance device 10 described in the first embodiment. The surgery assistance device 10F includes a two-dimensional image generation unit 12F instead of the two-dimensional image generation unit 12, and a composite image generation unit 14F instead of the composite image generation unit 14. The two-dimensional image generation unit 12F generates the two-dimensional image IM2F shown in FIG. 17. The composite image generation unit 14F generates and outputs the second screen W2F shown in FIG. 17.

The two-dimensional image IM2F includes three views, namely a PPV view, a PPVα view, and an OPVβ view. The PPV view includes only the elements described in b1, b2, b3, b4, and b7 in the first embodiment. The PPVα view includes only the elements described in b1, b2, b4, b5, and b6 in the first embodiment. The OPVβ view includes only the elements described in b1, b2, b7, b8, and b9 in the first embodiment. That is, in the seventh embodiment, the PPV view, the PPVα view, and the OPVβ view, which are displayed as a single view in the first embodiment, are each independently and individually displayed.

In this way, various modifications are possible to the form of the two-dimensional image IM2F. The surgery assistance device 10F according to the seventh embodiment is capable of achieving the same effects as those of the first embodiment described above.

### Eighth Embodiment

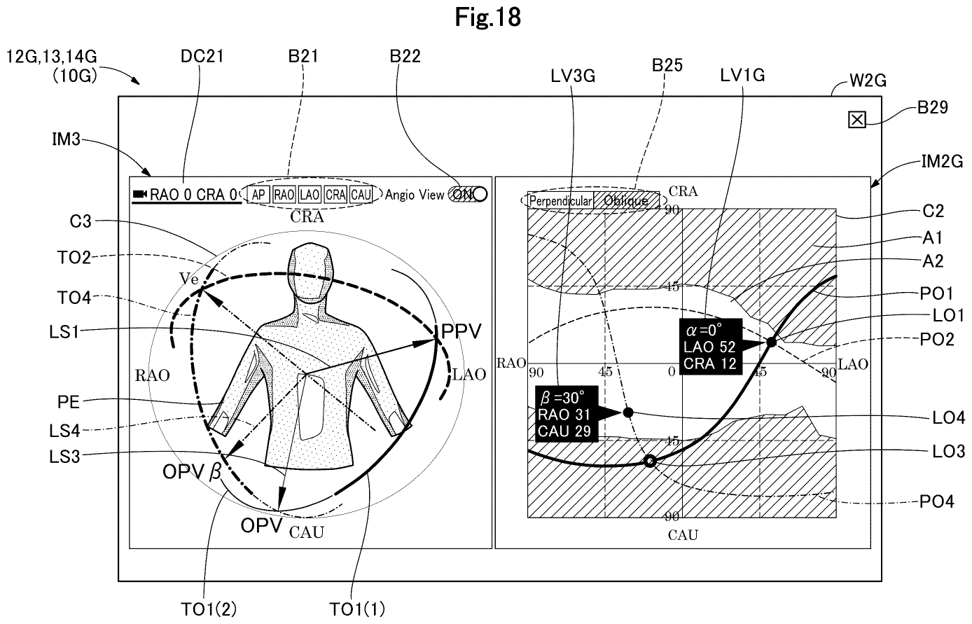

FIG. 18 is a diagram showing an example of a composite image according to an eighth embodiment. The surgery assistance system 1 according to the eighth embodiment includes a surgery assistance device 10G instead of the surgery assistance device 10 described in the first embodiment. The surgery assistance device 10G includes a two-dimensional image generation unit 12G instead of the two-dimensional image generation unit 12, and a composite image generation unit 14G instead of the composite image generation unit 14. The two-dimensional image generation unit 12G generates the two-dimensional image IM2G shown in FIG. 18. The composite image generation unit 14G generates and outputs the second screen W2G shown in FIG. 18.

The second screen W2G does not include the vertical position LV1 and the normal position LV3 described in the first embodiment. Instead, the two-dimensional image IM2G includes the vertical position LV1G and the normal position LV3G. The vertical position LV1G is displayed inside a balloon that extends from one of the first position LO1 and the second position LO2, which is represented by a black circle. The normal position LV3G is displayed inside a balloon that extends from one of the third position LO3 and the fourth position LO4, which is represented by a black circle.

In this way, various modifications are possible to the form of the two-dimensional image IM2G. The vertical position LV1G and the normal position LV3G may be included in the two-dimensional image IM2G rather than the composite image. The vertical position LV1G and the normal position LV3G may be included in both the composite image and the two-dimensional image IM2G. The surgery assistance device 10G according to the eighth embodiment is capable of achieving the same effects as those of the first embodiment described above.

### Modifications of Embodiments

The present disclosure is not limited to the embodiments described above, and may be implemented in various forms without departing from the gist of the present disclosure. For example, the following modifications are also possible.

### First Modification

The forms of the surgery assistance system 1 have been described in the first to eighth embodiments above. Various modifications are possible to the form of the surgery assistance system 1. For example, in the surgery assistance system 1, the surgery assistance device 10, or 10A to 10G may be connected to the radiation imaging device 20 and the like via the Internet. For example, the display device 30 may be a monitor or touch panel built into the surgery assistance device 10, or 10A to 10G. For example, the radiation imaging device 20 may include a second FPD that is placed in the normal direction of the FPD 21. For example, the surgery assistance system 1 may include another medical device or the like that is not illustrated. Examples of other medical devices include a CT device and an MRI device. In a case where other medical devices are included, the second screen W2 may include images acquired from the other medical devices.

### Second Modification

The forms of the surgery assistance device 10, and 10A to 10G have been described in the first to eighth embodiments above. Various modifications are possible to the form of the surgery assistance device 10, and 10A to 10G. For example, in the processing procedure described in FIG. 6, the execution order of the steps may be changed, at least some of the steps may be omitted, and other steps that have not been described may be executed.

For example, the calculation unit 11 may acquire the first image and the second image from a location other than a local folder of the surgery assistance device 10. Examples of other locations include a recording medium built into the radiation imaging device 20, an external device that is directly connected or connected over a network to the radiation imaging device 20, and an external recording medium. For example, the calculation unit 11 may determine the blood vessel axis vector by a different method to the method described in step S14. For example, instead of determining the blood vessel axis vector, the calculation unit 11 may calculate the blood vessel axis Ve by obtaining the start coordinates and end coordinates of the target blood vessel. For example, step S20 may be omitted. In this case, after completing step S14, the processing may directly transition to step S22.

For example, the first screen W1 is merely an example, and various modifications are possible. For example, the layout of each item on the screen, the presence or absence of the items, and the operation method of the screen can be changed as appropriate. For example, the second vector drawing button B123, the display panel DC12, and the display panel DC14 may be omitted. For example, the trajectory display button B11 may be omitted, and the processing may automatically proceed to the following step once vector drawing is complete. For example, at least one or more of the cancel button B121, the magnification button B124, the range movement button B125, and the redo button B126 may be omitted. For example, the first screen W1 may acquire touch operations made by a finger instead of mouse operations, or together with mouse operations.

For example, the second screen W2 is merely an example, and various modifications are possible. For example, the layout of each item on the screen, the presence or absence of the items, and the operation method of the screen can be changed as appropriate. For example, the two-dimensional image IM2 does not have to include the non-movable range A1 and the movable range A2. For example, the two-dimensional image IM2 does not have to include the mode specification button B25. For example, the two-dimensional image IM2 may include other items that have not been described above. For example, the three-dimensional image IM3 does not have to include the patient image PE. For example, the three-dimensional image IM3 does not have to include at least one or more of the display panel DC21, the viewpoint change buttons B21, and the head portion position fixing button B22. For example, the three-dimensional image IM3 may include other items that have not been described above. For example, in a case where the viewpoint change buttons B21 and the head portion position fixing button B22 are omitted, the viewpoint of the three-dimensional image IM3 may be fixed. For example, the second screen W2 may acquire touch operations made by a finger instead of mouse operations, or together with mouse operations.

In the embodiment described above, it has been assumed that, in the second screen W2, the blood vessel axis Ve passes through the origin of the three-dimensional coordinates C3. However, the origins of the blood vessel axis Ve and three-dimensional coordinates C3 do not necessarily have to coincide. Specifically, if the deviation between the blood vessel axis Ve and the origin of the three-dimensional coordinates C3 is within a predetermined error range, it can be considered that “the blood vessel axis Ve passes through the origin of the three-dimensional coordinates C3”.

In the embodiment described above, the first to fourth line segments LS1 to LS4 in the three-dimensional image IM3 are changed by operating the first to fourth positions LO1 to LO4 of the two-dimensional image IM2. In contrast, by operating the first to fourth line segments LS1 to LS4 of the three-dimensional image IM3, the first to fourth positions LO1 to LO4 of the two-dimensional image IM2 may be changed so as to follow the change in the positions of the first to fourth line segments LS1 to LS4. By operating one of the positions in the two-dimensional image IM2 and the line segments in the three-dimensional image IM3, the other can be made to follow.

### Third Modification

The forms of the surgery assistance devices 10, and 10A to 10G of the first to eighth embodiments described above, and the aspects of the first and second modifications described above may be combined as appropriate. For example, the elements described in the second to eighth embodiments may be combined to form a new second screen W2. For example, it may be possible to select the display mode of the second screen W2 from among the display modes of the second to eighth embodiments. In this case, for example, a mode is possible in which a setting is made using a setting screen, or a mode is possible in which a selection is made at the start of the processing of FIG. 6.

The present aspect has been described above based on the embodiments and the modifications. The embodiments of the aspect described above are intended to facilitate understanding of the present aspect, and do not limit the present aspect. The present aspect may be modified and improved without departing from the gist and the scope of the claims, and the present aspect includes equivalents thereof. If the technical features are not described as essential in the present specification, the technical features may be appropriately removed.

What is claimed is:

1. A surgery assistance device comprising:
circuitry configured to
generate, in a three-dimensional space, an image showing a range of placement positions of a flat panel detector (FPD) when a target blood vessel is imaged using the FPD, which can be moved along a surface of a virtual sphere having a predetermined radius and is centered at an origin of the three-dimensional space; and
generate a composite image, wherein
the circuitry
generates a two-dimensional image showing a placement position of the FPD in two-dimensional coordinates, the two-dimensional image including a first planar trajectory on which the placement position of the FPD is located in a first direction, which is perpendicular to an extending direction of the target blood vessel,
generates a three-dimensional image showing the placement position of the FPD) in three-dimensional coordinates, the three-dimensional image including a blood vessel axis that represents the extending direction of the target blood vessel as a line segment extending from the origin of the three-dimensional coordinates, and a first three-dimensional trajectory that shows the first planar trajectory as a circumference of a circle having a radius of a predetermined length from the origin of the three-dimensional coordinates, and
generates and outputs the composite image, in which the two-dimensional image and the three-dimensional image have been combined.

2. The surgery assistance device according to claim 1, wherein the circuitry
generates the two-dimensional image further including a first position, which is an arbitrary placement position of the FPD) on the first planar trajectory, and
generates the three-dimensional image further including a first line segment, in which the first position on the first planar trajectory is represented by a line segment extending from the origin of the three-dimensional coordinates to the first position.

3. The surgery assistance device according to claim 2, wherein
the first position in the two-dimensional image and the first line segment in the three-dimensional image are linked to each other.

4. The surgery assistance device according to claim 2, wherein the circuitry
generates the two-dimensional image further including a second planar trajectory, in which the placement position of the FPD is located in a second direction, which is on a first virtual plane that passes through the extending direction of the target blood vessel and a virtual line segment extending from the origin of the three-dimensional space to the first position, and
generates the three-dimensional image further including a second three-dimensional trajectory that shows the second planar trajectory as a circumference of a circle that includes the first virtual plane, and also having a radius of a predetermined length from the origin of the three-dimensional coordinates.

5. The surgery assistance device according to claim 4, wherein the circuitry
generates the two-dimensional image further including a second position, which is an arbitrary placement position of the FPD on the second planar trajectory, and
generates the three-dimensional image further including a second line segment, in which the second position on the second planar trajectory is represented by a line segment extending from the origin of the three-dimensional coordinates to the second position.

6. The surgery assistance device according to claim 5, wherein
the second position in the two-dimensional image and the second line segment in the three-dimensional image are linked to each other.

7. The surgery assistance device according to claim 2, wherein the circuitry
generates the two-dimensional image further including a third position, being a position separated from the first position on the first planar trajectory, the third position being on a second virtual plane obtained by rotating the first virtual plane, which passes through the extending direction of the target blood vessel and a virtual line segment extending from the origin of the three-dimensional space to the first position, by 90°around the extending direction of the target blood vessel, and
generates the three-dimensional image further including a third line segment, in which the third position on the first planar trajectory is represented by a line segment extending from the origin of the three-dimensional coordinates to the third position.

8. The surgery assistance device according to claim 7, wherein
the third position in the two-dimensional image and the third line segment in the three-dimensional image are linked to each other.

9. The surgery assistance device according to claim 7, wherein the circuitry
generates the two-dimensional image further including a fourth planar trajectory, in which a placement position of the FPD is located in a fourth direction, which is on the second virtual plane, and
generates the three-dimensional image further including a fourth three-dimensional trajectory that shows the fourth planar trajectory as a circumference of a circle that includes the second virtual plane, and also having a radius of a predetermined length from the origin of the three-dimensional coordinates.

10. The surgery assistance device according to claim 9, wherein the circuitry
generates the two-dimensional image further including a fourth position, which is an arbitrary placement position of the FPD on the fourth planar trajectory, and
generates the three-dimensional image further including a fourth line segment, in which the fourth position on the fourth planar trajectory is represented by a line segment extending from the origin of the three-dimensional coordinates to the fourth position.

11. The surgery assistance device according to claim 10, wherein
the fourth position in the two-dimensional image and the fourth line segment in the three-dimensional image are linked to each other.

12. The surgery assistance device according to claim 1, wherein
the circuitry changes a viewpoint of the three-dimensional image as a result of the three-dimensional image being rotated according to an operation by an operator.

13. The surgery assistance device according to claim 12, wherein
the circuitry generates the three-dimensional image including a patient image, which is an image of a patient including the target blood vessel, and
the three-dimensional image is rotated in a state where a head portion of the patient is fixed in a predetermined direction in the three-dimensional image.

14. The surgery assistance device according to claim 2, wherein
the composite image further includes a character string indicating a position of the FPD for placing the FPD in the first position.

15. The surgery assistance device according to claim 5, wherein
the composite image further includes a character string indicating a position of the FPD for placing the FPD in the second position.

16. The surgery assistance device according to claim 7, wherein
the composite image further includes a character string indicating a position of the FPD for placing the FPD in the third position.

17. The surgery assistance device according to claim 10, wherein
the composite image further includes a character string indicating a position of the FPD for placing the FPD in the fourth position.

18. A method of assisting a surgery including a process of capturing, in a three-dimensional space, a target blood vessel using a flat panel detector (FPD) that can be moved along a surface of a virtual sphere having a predetermined radius and is centered at an origin of the three-dimensional space, the method comprising:
generating a two-dimensional image showing a range of placement positions of the FPD;
generating a three-dimensional image showing a range of placement positions of the FPD; and
generating a composite image, wherein
the two-dimensional image is generated showing a placement position of the FPD in two-dimensional coordinates, the two-dimensional image including a first planar trajectory on which the placement position of the FPD is located in a first direction, which is perpendicular to an extending direction of the target blood vessel,
the three-dimensional image is generated showing the placement position of the FPD in three-dimensional coordinates, the three-dimensional image including a blood vessel axis that represents the extending direction of the target blood vessel as a line segment extending from the origin of the three-dimensional coordinates, and a first three-dimensional trajectory that shows the first planar trajectory as a circumference of a circle having a radius of a predetermined length from the origin of the three-dimensional coordinates, and
the composite image, in which the two-dimensional image and the three-dimensional image have been combined, is generated and output.

19. A non-transitory computer-readable medium storing thereon a program for assisting a surgery including a process of capturing, in a three-dimensional space, a target blood vessel using a flat panel detector (FPD) that can be moved along a surface of a virtual sphere having a predetermined radius and is centered at an origin of the three-dimensional space, the program causing a computer to execute:
generating a two-dimensional image showing a range of placement positions of the FPD;
generating a three-dimensional image showing a range of placement positions of the FPD; and
generating a composite image, wherein
the two-dimensional image is generated showing a placement position of the FPD in two-dimensional coordinates, the two-dimensional image including a first planar trajectory on which the placement position of the FPD is located in a first direction, which is perpendicular to an extending direction of the target blood vessel,
the three-dimensional image is generated showing the placement position of the FPD in three-dimensional coordinates, the three-dimensional image including a blood vessel axis that represents the extending direction of the target blood vessel as a line segment extending from the origin of the three-dimensional coordinates, and a first three-dimensional trajectory that shows the first planar trajectory as a circumference of a circle having a radius of a predetermined length from the origin of the three-dimensional coordinates, and
the composite image, in which the two-dimensional image and the three-dimensional image have been combined, is generated and output.

20. The non-transitory computer-readable medium according to claim 19, wherein
the two-dimensional image further includes a first position, which is an arbitrary placement position of the FPD on the first planar trajectory, and
the three-dimensional image further includes a first line segment, in which the first position on the first planar trajectory is represented by a line segment extending from the origin of the three-dimensional coordinates to the first position.
